(19) United States
(12) Patent Application Publication
(10) Pub. No.: US 2008/0063681 A1
(43) Pub. Date: Mar. 13, 2008

(54) Therapeutic bone replacement material

(75) Inventors: Bruce Simon, Mountain Lakes, NJ (US); Joshua Simon, Rockaway, NJ (US)

Correspondence Address: HARNESS, DICKEY & PIERCE, P.L.C., P.O. BOX 828, BLOOMFIELD HILLS, MI, 48303, US

(73) Assignee: EBI, L.P., Parsippany, NJ (US)

(21) Appl. No.: 11/519,168

(22) Filed: Sep. 11, 2006

(51) Int. Cl. A61K 33/42; A61K 31/65; A61F 2/02

(52) U.S. Cl. 424/423; 514/153; 424/602

(57) ABSTRACT

A biodegradable bone replacement material for the treatment of bone defects and delivery of antibiotic compounds, particularly for treating bone infections, comprising calcium sulfate hemihydrate, calcium sulfate dihydrate, an antibiotic mixture comprising a tetracycline compound and an ansamycin compound. Preferably, the ratio of calcium sulfate hemihydrate to calcium sulfate dihydrate is from 1:1 to about 3:1. Methods for treating, repairing or augmenting an osseous defect using the bone replacement material are also provided.

### BACKGROUND

The present disclosure relates to bone replacement materials including graft and bone void filler materials.

Osteomyelitis is medically characterized as an infection of the osseous tissue. Bacterial infection of the bone occurs most often by a haematogenous route. Initial entry of infectious pathogens into the metaphysis of long bones is typically through tortuous capillary loops that are prone to thrombosis, allowing bacterial seeding. (Thirty percent of cases have recent history of blunt trauma and infection, producing an acute inflammatory response causing edema within the bone and soft tissues.) This can progress into the bone marrow and through the cortical bone via the Haversian canals. Pus can then form within the cancellous bone and beneath the periosteum from where it may break into the soft tissues and extend to the surface as a sinus tract. Subperiosteal pus can strip off the overlying periosteum causing bone death. Antibiotic therapy is continued for at least three weeks after surgery. In other cases, infection of an orthopedic prosthesis requires surgical removal with debridement of the infected tissue surrounding the area. A new prosthesis may be implanted in the same operation, or delayed until the infection has resolved, depending on its severity. Resistant chronic osteomyelitis may result in amputation and can threaten life through seeding of the microorganisms to cardiac valves, the lungs, and the brain.

The open space left by the removed bone tissue may be filled with bone graft or by packing material to promote the growth of new bone tissue. Antibiotic formulations of polymethylmethacrylate (PMMA) have been employed as antiseptic bone cement and as beads either free or attached to a wire which is used for percutaneous removal. See, H. W. Bucholz, et al, Chiburg, 43, 46 (1970). PMMA is not biodegradable and must be removed requiring a second surgery. Compositions of calcium sulfate hemihydrate have been used to manufacture vehicles for medicinal delivery, however, due to its rapid resorption rate and very long setting times, calcium sulfate hemihydrate alone is not a suitable candidate for a bone replacement material for use intra-operatively. Calcium sulfate dihydrate implants suffer from excessively shortened setting times making it impractical for intra-operative use. Therefore, there is a need for bone void filler or graft material that can prevent the onset of chronic osteomyelitis and still provide an osteoconductive scaffold for osteogenesis and bone remodeling.

### SUMMARY

The present technology provides a bone replacement material for use in filling bone voids, and repairing and augmenting bone defects, in patients with a bone infection or those who are susceptible to having a bone infection and are in need of orthopedic or oral/maxillofacial surgery. The bone replacement material comprises calcium sulfate hemihydrate, calcium sulfate dihydrate and an antibiotic mixture comprising a tetracycline antibiotic and an ansamycin antibiotic. In various embodiments, the ratio of calcium sulfate hemihydrate to calcium sulfate dihydrate is from about 1:1 to about 3:1.

Bone replacement materials among those described herein provide advantages over implantable calcium ceramics containing antibiotics known in the art. Such advantages include one or more of increased osteoconductivity, increased selectivity for the therapeutic treatment of persistent bone infections (i.e., chronic osteomyelitis), increased stability, and reduced side effects. In various embodiments, the technology provides the ability to add the tetracycline antibiotic along with an ansamysin antibiotic to a calcium sulfate ceramic to obtain a bone replacement material having clinically acceptable setting times for intra-operative surgical use. In various embodiments, the mixture and ratios of calcium sulfate hemihydrate and dihydrate and antibiotic results in a stable formulation which allows the antibiotics to be released in a controlled manner for in situ delivery at the site of infection, particularly within bone defects. By maintaining a desired local level of the two antibiotic classes in the bone defect, the implantable bone replacement material achieves physiological effects including broad-spectrum antibiotic activity, and an osteoconductive framework to initiate bone growth and remodeling within the bone defect. Further areas of applicability will become apparent from the description provided herein.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. The following definitions and non-limiting guidelines must be considered in reviewing the description of the technology set forth herein.

The headings (such as “Introduction” and “Summary,”) and sub-headings (such as “Bone Graft Material”) used herein are intended only for general organization of topics within the disclosure of this technology, and are not intended to limit the disclosure of the technology or any aspect thereof. In particular, subject matter disclosed in the “Background” may include novel technology, and may not constitute a recitation of prior art. Subject matter disclosed in the “Summary” is not an exhaustive or complete disclosure of the entire scope of the technology or any embodiments thereof.

The citation of references herein and during prosecution of applications regarding this technology does not constitute an admission that those references are prior art or have any relevance to the patentability of the technology disclosed herein. Any discussion of the content of references cited in the Introduction is intended merely to provide a general summary of assertions made by the authors of the references, and does not constitute an admission as to the accuracy of the content of such references. All references cited in the Description section of this specification are hereby incorporated by reference in their entirety.

The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific Examples are provided for illustrative purposes of how to make, use and practice the compositions and methods of this technology and, unless explicitly stated otherwise, are not intended to be a representation that given embodiments of this technology have, or have not, been made or tested.

As used herein, the words “preferred” and “preferably” refer to embodiments that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

As used herein, the word “include,” and its variants, is intended to be non-limiting, such that recitation of an item or items in a list is not to the exclusion of other like items that may also be useful in the materials, compositions, devices, and methods of this technology. Unless otherwise indicated, all percentages are by weight of the composition.

### Bone Graft Materials

In various embodiments, a bone replacement material including materials used to fill bone voids and other defects of osseous tissue in accordance with the present disclosure comprises calcium sulfate hemihydrate, calcium sulfate dihydrate, and an antibiotic mixture of at least one tetracycline antibiotic and at least one ansamycin antibiotic, preferably wherein the ratio of the calcium sulfate hemihydrate to the calcium sulfate dihydrate being from about 1:1 to about 3:1. The bone replacement material can optionally contain radiopaque agents and osteoinductive agents.

In various embodiments, the bone replacement material comprises an inorganic calcium sulfate ceramic comprising calcium sulfate hemihydrate and calcium sulfate dihydrate. Calcium sulfate hemihydrate CaSO₄.½H₂O is commonly known as Plaster of Paris. Calcium sulfate hemihydrate exists in two forms, alpha form and beta form. The alpha form consists of compact, well formed, and transparent large primary particles. The beta form has rhombohedral structure and consists of rugged secondary particles made up of extremely small crystals. Plaster of Paris is made from calcium sulfate dihydrate (gypsum) through dehydration. The gypsum is ground and heated until about 75% of the water is gone and CaSO₄.½H₂O is obtained.

2(CaSO₄.2H₂O)→2(CaSO₄.½H₂O)+3H₂ (1)

2(CaSO₄.½H₂O)+3H₂O→2(CaSO₄.2H₂O)+heat (2)

The calcium sulfate ceramic used in the bone replacement material is preferably α-calcium sulfate hemihydrate and/or β-calcium sulfate hemihydrate and calcium sulfate dihydrate. Medical grade calcium sulfate hemihydrate is commercially available as BonePlast™ (Biomet Irvine, Inc., Irvine Calif., USA). Calcium sulfate dihydrate is commercially available as Calcigen S™ (Biomet Orthopedics Inc., Warsaw, Ind., USA).

In various embodiments, addition of calcium sulfate dihydrate to calcium sulfate hemihydrate will decrease the setting time because the nucleation time is eliminated. Without being bound to theory, crystal growing can start directly on the calcium sulfate dihydrate particles which can act as an accelerator. The setting rate of the calcium sulfate ceramic is largely dependant on the ratios of the hemihydrate to dihydrate. In embodiments of the present technology, differing ratios of calcium sulfate hemihydrate to calcium sulfate dihydrate can be manipulated to form hardened calcium sulfate ceramics with varying setting times containing therapeutic antibiotics specific for treating and preventing bacterial infections of the bone, and in some embodiments, so as to produce formulations having clinically useful setting times.

Thus, in various embodiments, the amount of calcium sulfate hemihydrate to calcium sulfate dihydrate depends on the intended use and the setting time needed i.e. the time elapsed between the time the dried components are wetted with an aqueous solution and formed into a flowable or injectable slurry and the time when the slurry hardens into a solid. In some embodiments, the bone replacement material is prepared to have a faster setting time by adding more calcium sulfate dihydrate to the composition; conversely, adding greater quantities of calcium sulfate hemihydrate can retard the setting time. It is thus preferred in some embodiments to adjust the ratios of calcium sulfate hemihydrate and calcium sulfate dihydrate between 1:1 to 3:1, particularly if conditions during surgery require a rapid implantation of the bone replacement material or delays are present that require keeping the bone replacement material in a liquid/semi-liquid state longer.

In various embodiments, the ratio of calcium sulfate hemihydrate to calcium sulfate dihydrate can range from about 1:1, from about 1.5:1, from about 2.0:1, or from about 2.5:1 to about 3:1. In various embodiments, the bone replacement material contains greater than or equal to about 50%, 55%, 60%, 65%, 70%, 75%, or 80% of calcium sulfate ceramic.

The calcium sulfate ceramic can be prepared as a mixture of anhydrous powders of calcium sulfate hemihydrate and calcium sulfate dihydrate and then wetted and hydrated with the mixing solution. In some embodiments, the antibiotics can be added to the calcium sulfate ceramic as powders or in solution. The volume of mixing solution can be selected to provide the composition with a desired consistency and setting time. In various embodiments, the mixing solution includes an aqueous solution having any water soluble salts of polyfunctional carboxylic acids containing 2 to about 10 carbons, preferably citrates, and/or dibasic phosphate salts as described in U.S. Pat. No. 5,281,265. In some embodiments, the mixing solution can be a solution comprising sterile water, potassium citrate and sodium phosphate. In various embodiments, the mixing solution is greater than about 15%, 20%, 22% 25% 27% 30% 33% 36 % 40% 45%, 50%, or 55% of the bone replacement material.

In various embodiments, the bone replacement material consists essentially of calcium sulfate hemihydrate, calcium sulfate dihydrate, and at least one antibiotic derived from the tetracycline class and at least one antibiotic derived from the ansamycin class of antibiotics. In various embodiments, the bone replacement material is essentially free of complexing agents, plasticizers, for example cellulose containing agents such as methyl cellulose and its derivatives, binders and/or matrix polymers. In various embodiments, the bone replacement material does not contain any significant amount of material that would affect the viscosity or setting times provided by biopolymers, for example, collagen, gelatin, fibrinogen, hydrolytic enzymes, calcium stearate, zinc undecylenate, magnesium palmitate, sodium laurate, calcium napthenate, calcium oleate, lauryl ammonium sulfate, hyaluronic acid, acidic proteins, vinyl alcohols, stearic acids, polynucleotides, polyglutamic acid, polyaspartic acid, pamoic acid, dextran, dextran sulfate, pentosan polysulfate, glycoaminoglycans, chondroitin sulfate, and the like.

### Antibiotics

In various embodiments, the bone replacement material comprises two or more antibiotics that can be useful in treating acute or chronic bone infections. In some embodiments, at least one antibiotic from the tetracycline class and at least one antibiotic from the ansamycin class of antibiotics are mixed with the calcium sulfate ceramic. In some embodiments, preferred antibiotics from each class include minocycline and rifampin. In some embodiments, sterile minocycline and rifampin in powdered form can be admixed with the other powders of the bone replacement material including the calcium sulfate hemihydrate and the calcium sulfate dihydrate prior to the addition of the mixing solution.

Ansamycin antibiotics are macrocyclic molecules composed of a benzoic or naphthalenic chromophore bridged by an aliphatic polyketide chain that terminates at the chromophore with an amide linkage. The aromatic moiety is derived from a 3-amino-5-hydroxybenzoic acid (AHBA) primer unit which is activated by a nonribosomal peptide synthetase-like mechanism and processed via addition of methylmalonyl and malonyl units by a multimodular polyketide synthase.

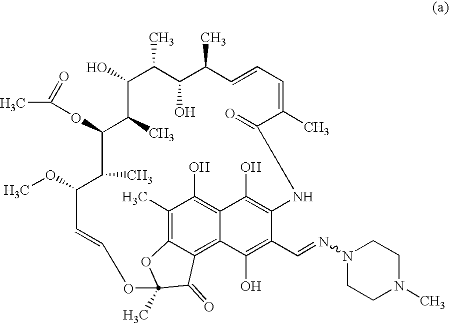

Rifampin is a semisynthetic derivative of rifamycin, a macrocyclic antibiotic compound produced by the mold Streptomyces mediterranic. Rifampin inhibits bacterial DNA-dependent RNA polymerase activity and is bactericidal in nature. Rifampin is a zwitterion that is soluble in acidic aqueous solutions, is even more soluble in organic solvents, and displays exceptional diffusion through lipids. (Rifampin is commercially available from Novartis, East Hanover, N.J., USA)

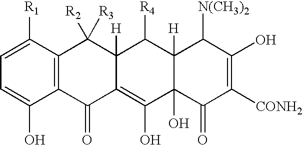

Minocycline is a semisynthetic antibiotic derived from tetracycline. It is primarily bacteriostatic and exerts its antimicrobial effect by inhibiting protein synthesis. Minocycline is commercially available as the hydrochloride salt which occurs as a yellow, crystalline powder and is soluble in water and slightly soluble in alcohol. (Minocycline is commercially available from Triax Pharmaceuticals Mountain Lakes, N.J., USA.)

In various embodiments, the mixture of tetracycline and ansamycin antibiotics provides a broad spectrum of activity against organisms that cause orthopedic, neurosurgical and oral and maxillofacial surgical related infections, including Staphylococcus epidermidis, Staphylococcus aureus, streptococci, mycobacteria, corynebacteria, gram-negative bacilli, and Candida. As used herein, a bolus of antibiotic can release upon implantation of the bone replacement material comprising (a) calcium sulfate hemihydrate, (b) calcium sulfate dihydrate; and (c) an antibiotic mixture comprising a tetracycline compound and an ansamycin compound wherein the ratio of calcium sulfate hemihydrate to calcium sulfate dihydrate is from about 1:1 to about 3:1. In some embodiments, approximately 80% of the antibiotic is released from the bone replacement material within two days providing therapeutic levels of antibiotics when used to combat a reoccurring infection such as chronic osteomyelitis or when conventional antibiotics used to treat osseous infections such as gentamycin, tobramycin, cefazolin and vancomycin are ineffective. In some embodiments, the release time is greater than or equal to three days, alternatively, greater than or equal to 5, 7, 10 or 15 days. As referred to herein, “release time” is the time required for at least about 90% of the antibiotic to be released from the composition.

In various embodiments, the tetracycline antibiotic and the ansamycin antibiotic is added to the calcium sulfate ceramic in concentrations that are non-toxic to the recipient and at concentrations having an effective bactericidal affect on the infectious agent being eradicated. In some embodiments, the bone replacement material comprises from about 0.1% to about 3%, or greater than or equal to 0.01%, 0.1%, 0.5%, 1.5%, 2.0% 2.5%, 2.9%, of a tetracycline antibiotic. In some embodiments, the bone replacement material comprises from about 0.1% to about 0.01%, or greater than or equal to 0.1%, 0.5%, 1.5%, 2.0% 2.5% or 2.9%, of an ansamycin antibiotic.

### Osteoinductive Agents

In various embodiments, the bone replacement material can optionally include one or more osteoinductive agents. In some embodiments of the present technology, the osteoinductive agent includes any one or more of demineralized bone matrix (commercially available as Accell® DBM100, Citagenix, Quebec CA; Grafton®, Osteotech, New Jersey USA and Intergro®, Interpore, California USA); BMP's, such as BMP2, BMP3 (Osteogenin), BMP3B (Growth and Differentiation Factor (GDF) 10), BMP4, BMP5, BMP6 (Vgr1), BMP7 (Osteogenic Protein (OP) 1), BMP8 (OP2), BMP8B (OP3), BMP9 (GDF2), BMP10, BMP11, BMP12, BMP13, BMP14, BMP15 and BMP16; insulin-like growth factor (IGF-1 & 2); transforming growth factor betal (TGF-β1); platelet derived growth factor (PDGF); beta-fibroblast growth factor (β-FGF); vascular endothelial growth factor (VEGF); osteocalcin, osteopontin; and other blood derived proteins. Although a great majority, if not all of these osteoinductive factors can be found in DBM, there may be other unidentified osteoinductive factors present in DBM. Bone replacement materials of the present technology can also include osteoinductive agents that are isolated from natural sources or purified by recombinant methods. In various embodiments, the bone replacement material includes between about 0% to about 15% (wt. %) of the one or more optional osteoinductive agents, such as between about 1% to about 13%, or between about 3% to about 10%, or about 5% to about 8% (wt. %) of the bone replacement material.

In some embodiments, the bone replacement material can be radiographically visualized using a radiopaque substance. The bone replacement material can also optionally include at least one radiopaque marker for example, barium sulfate, barium fluoride, barium polyacrylate, iodipamide, bismuth, lead, mercury, uranium, silver, gold, zirconium, titanium dioxide, chromium oxide.

### Methods of Use

In various embodiments, the powders of calcium sulfate hemihydrate and calcium sulfate dihydrate are mixed prior to the addition of the mixing solution and the antibiotic mixture comprising one or more tetracycline antibiotics and at least one ansamycin antibiotics. In some embodiments, the calcium sulfate ceramic comprising mixtures of calcium sulfate hemihydrate and dihydrate preferably in ratios varying from 1:1 to about 3:1, can be mixed with the selection of antibiotics, for example minocycline and rifampin to form the composition.

In various embodiments, the surgeon performing the osteotomy or debridement of the infected bone tissue or defect can intra-operatively mix the calcium sulfate powders, add the antibiotic mixture to the powder mix, optionally add the demineralized bone matrix or other osteoinductive materials, and apply the appropriate quantity of mixing solution (such as an acidic solution comprising sterile water and potassium citrate and sodium phosphate) to obtain a flowable paste. Alternatively, the powders comprising the calcium sulfate hemihydrate, calcium sulfate dihydrate, and the antibiotic mixture can be wetted with the patient's blood or other bodily fluid, such as bone marrow aspirate. The composition can be a conforming material having a paste like consistency or contacted with a smaller volume of mixing solution to form a material having a putty like consistency that can be applied manually into the defect site, for example, to fill in the cracks and voids after debridement of unwanted cells and other tissues. In some embodiments, the paste comprising the bone replacement material can be put into a sterile syringe and injected into the defect site, for example with an 18 gauge syringe. In a preferred embodiment, the surgeon or technician can prepare the bone replacement material intra-operatively, thereby adjusting the appropriate formulation of the material for the specific application, infection, bone type, and surgical technique performed. As defined herein, the term “intra-operative” refers to preparatory procedures occurring during the course of surgery. In various embodiments, the bone replacement material is capable of setting to hardness in about 3 to about 12 minutes, for example greater than 2, 5, 7, 9, or 11 minutes, and/or less than 13, 11, 9, 7 or 4 minutes.

In some embodiments, the bone replacement material is compressed, molded or extruded into any pharmaceutically acceptable shape for implantation into a defect site. In some embodiments, the bone replacement material can be molded into the shape of pellets, beads, granules and any other desired shape. The pellets or beads can then be implanted into the defect site and then covered with skin grafts or tissue flaps. The porous ceramic composite can be placed in proximity and/or into the defect site with a surgical tool or with manual manipulation by the surgical operator.

The bone replacement material can be utilized in a wide variety of orthopedic, neurosurgical and oral and maxillofacial surgical procedures to prevent osteomyelitis and other bacterial and yeast infections of the bone in susceptible patients, for example, those patients with prior history of acute or chronic osteomyelitis, those undergoing an infection other than osteomyelitis, such as a bacteremia, patients with diabetes, and those who are undergoing immune suppression therapy. Bone replacement materials according to the present technology can be used in susceptible patients in need of reparation of bone defects including, simple and compound fractures and non-unions, external and internal fixations, joint reconstructions such as arthrodesis, general arthroplasty, cup arthroplasty of the hip, femoral and humeral head replacement, femoral head surface replacement and total joint replacement, repairs of the vertebral column including spinal fusion and internal fixation, tumor surgery, e.g. deficit filling, discectomy, laminectomy, excision of spinal cord tumors, anterior cervical and thoracic operations, repair of spinal injuries, scoliosis, lordosis and kyphosis treatments, intermaxillary fixation of fractures, mentoplasty, temporomandibular joint replacement, alveolar ridge augmentation and reconstruction, inlay bone grafts, periodontal bone replacement, implant placement and revision, sinus lifts, etc. As used herein, “bone defect(s)” or “injury sites” and variants thereof, encompass bone imperfections caused by congenital defect, trauma, disease, decay or surgical intervention, and the desired repair can be for cosmetic or therapeutic reasons.

In various embodiments, the bone replacement material includes a three-dimensional object pre-selected for the particular bone defect in need of repair. Once the defect or foci of infection has been washed and debrided, including the removal of dead bone and associated sequelae, the bone replacement material can be placed into the defect site with a surgical tool, or alternatively manually placed by the surgical operator and if needed, affixed using staples, sutures, or biological bone cement. In some embodiments, the bone replacement material can be hydrated with the mixing solution, optionally containing demineralized bone matrix to form a coating for an orthopedic device or implant, for example, the back of a tibial tray or acetabular cup. In some embodiments, the surgical operator can match the contour of the bone replacement material with the contour of the bone defect site or implantable prosthesis, for example an acetabular cup. In various embodiments, the implanted bone replacement materials after debridement can be covered with tissue flaps or skin grafts from autogenous, allogeneic or synthetic sources which can serve in guided tissue regeneration or as barrier materials.

In some embodiments, the ceramic implant can be used to augment a defect site. In some embodiments, a defect site includes the femur above the patella. In such a femur defect, the bone replacement materials of the present technology can augment implants placed in these kinds of high load and stress sites to provide supplemental strength. New bone will grow into and around the implant and replace the porous ceramic body, or grow within and around the implant and the host tissue. In some embodiments, the bone replacement material of the present technology can also be used to augment defect sites resulting from surgical intervention. When the bone replacement material is coated onto and inserted into a prosthetic device, stability and longevity of the orthopedic device may be enhanced, by incorporating the patient's natural bone as a means for support.

In some embodiments, due to the destructive nature of chronic osteomyelitis, substantial bone may have decayed and is removed. Structural integrity and load bearing capabilities of the affected bone may require the use of prosthetic devices, such as rods and pins to add additional support to the treated bone, as in the case of hip arthroplasty, it may be necessary to prepare a lumen or tunnel within the femur to provide space for the femoral stem implant. The femoral stem implant can be secured in the tunnel with a surgical fixative, but added benefits can be achieved if the space between the tunnel and the femoral implant were filled with the present bone replacement material providing a controlled release of antibiotics to prevent infection occurring or reoccurring, and an osteoinductive and osteoconductive framework to secure the implant.

### Kits

In various embodiments, kits comprise sterile components of calcium sulfate hemihydrate and calcium sulfate dihydrate, an antibiotic mixture comprising a tetracycline compound and an ansamysin compound, and mixing solution, in separate containers. In some embodiments instructions on how to prepare various bone replacement materials with varying setting times are also included.

Embodiments of the present technology are further illustrated through the following non-limiting example.

### EXAMPLES

Mixtures of BonePlast™ (Biomet Irvine, Inc., Irvine, Calif., USA)—CaSO₄ Hemihydrate, (Hemi) and Calcigen S™ (Biomet Orthopedics Inc., Warsaw, Ind., USA)—CaSO₄ Dihydrate (Di) (mixtures of Hemi and Di are otherwise known as CaSO₄) in different ratios are manually combined with minocycline and/or rifampin, and mixed with 5 ml of an acid mixing solution comprising sterile water, potassium citrate and sodium phosphate measured using a Becton-Dickinson 10 ml syringe. The acid setting solution is commercially available for sale with Calcigen S™. A series of compositions are prepared, having differing compositions as follows: Group 1, comprising 10 g CaSO₄, 62.5 mg minocycline, and 62.5 mg rifampin; and Group 2, comprising 10 mg CaSO₄, 12.5 mg minocycline, and 12.5 mg rifampin. Within each group, individual compositions are made having varying ratios of hemihydrate: dehydrate, as follows: 0:100, 10:90, 20:80, 30:70, 50:50, 60:40, 70:30, 80:20 and 100:0.

All powder groups are poured into BonePlast trays at normal room temperature, 25° C. and poured onto the powder component. Using a plastic spatula, the components are stirred together for 2 minutes to produce a homogenous paste. The paste is then applied on hollow circular regions of BonePlast trays to make roughly 20-25 pellets. The material is allowed to set to form composite beads (6-mm diameter).

What is claimed is:

1. A biodegradable bone replacement material comprising (a) calcium sulfate hemihydrate, (b) calcium sulfate dihydrate, (c) an antibiotic mixture comprising a tetracycline compound and an ansamycin compound, and (d) a mixing solution, wherein the ratio of (a) to (b) is from about 1:1 to about 3:1.

2. A bone replacement material according to claim 1, wherein the mixing solution is selected from the group consisting of citrates, tartrates, dibasic phosphate salts, and mixtures thereof.

3. A bone replacement material according to claim 2, wherein the mixing solution comprises potassium citrate and sodium phosphate.

4. The bone replacement material according to claim 2, wherein the tetracycline compound is selected from the group consisting of chlortetracycline, oxytetracycline, doxycycline, clomocycline, demeclocycline, guamecycline, lymecycline, meclocycline, tetracycline, penimepicycline, pipacycline, rolitetracycline, sancycline, minocycline, methacycline, and pharmaceutically acceptable salts, esters, and enantiomers thereof.

5. The bone replacement material according to claim 4, wherein the tetracycline compound is minocycline.

6. The bone replacement material according to claim 1, wherein the ansamycin compound is selected from the group consisting of geldanamycin, rifamide, rifampin. rifamycin, rifapentine, and rifaximin and pharmaceutically acceptable salts, esters, and enantioners thereof.

7. The bone replacement material according to claim 6, wherein the ansamysin compound is rifampin.

8. The bone replacement material according to claim 1, wherein the antibiotic mixture has a greater resorption rate in vivo than the calcium sulfate hemihydrate and calcium sulfate dihydrate.

9. The bone replacement material according to claim 8, wherein the antibiotic mixture has a release time of greater than or equal to three days.

10. The bone replacement material according to claim 1, wherein the bone replacement material further comprises an osteoinductive agent.

11. The bone replacement material according to claim 10, wherein the osteoinductive agent is selected from the group consisting of demineralized bone, demineralized bone matrix, bone morphogenetic proteins (BMP 1-17), insulin-like growth factor (IGF-1 & 2), transforming growth factor betal (TGF-β1), platelet derived growth factor (PDGF), beta-fibroblast growth factor (P-FGF), vascular endothelial growth factor (VEGF), osteocalcin, osteopontin, and combinations thereof.

12. The bone replacement material according to claim 1, wherein the material further comprises a radiopaque marker.

13. The bone replacement material according to claim 12, wherein the radiopaque marker is selected from the group consisting of barium sulfate, barium fluoride, barium polyacrylate, iodipamide, bismuth, lead, mercury, uranium, silver, gold, zirconium, titanium dioxide, chromium oxide, and combinations thereof.

14. The bone replacement material according to claim 1, wherein the material is in the form selected from the group consisting of a pellet, a bead, a granule, a powder, a chip, and a shaped object.

15. A bone void filler comprising the bone replacement material in accordance with claim 1.

16. A dental prosthesis comprising the bone replacement material in accordance with claim 1.

17. An orthopedic prosthesis comprising the bone replacement material in accordance with claim 1.

18. A method of treating, repairing and augmenting a bone defect in osseous tissue, comprising applying to the site of the defect a bone replacement material, said material comprising
(a) calcium sulfate hemihydrate;
(b) calcium sulfate dihydrate;
(c) an antibiotic mixture comprising a tetracycline compound and an ansamycin compound; and
(d) a mixing solution,
wherein the ratio of (a) to (b) is from 1:1 to 3:1.

19. A method according to claim 18, wherein the mixing solution is selected from the group consisting of citrates, tartrates, dibasic phosphate salts, and mixtures thereof.

20. A method according to claim 18, wherein the antibiotic mixture comprises rifampin and minocycline.

21. A method according to claim 18, wherein said material further comprises one or more osteoinductive agents.

22. A method according to claim 21, wherein said osteoinductive agent is selected from the group consisting of demineralized bone, demineralized bone matrix, bone morphogenetic proteins (BMP 1-17), insulin-like growth factor (IGF-1 & 2), transforming growth factor betal (TGF-β1), platelet derived growth factor (PDGF), beta-fibroblast growth factor (P-FGF), vascular endothelial growth factor (VEGF), osteocalcin, osteopontin, and combinations thereof.

23. A kit containing sterile components comprising calcium sulfate hemihydrate and calcium sulfate dihydrate, and instructions for use thereof to prepare a bone replacement material by mixing the calcium sulfate hemihydrate and the calcium sulfate dihydrate with an antibiotic mixture and a mixing solution.

24. A kit according to claim 23, further comprising an antibiotic mixture comprising a tetracycline compound and an ansamycin compound.

25. A kit according to claim 23, further comprising a mixing solution.
